(19) United States
(12) Patent Application Publication
(10) Pub. No.: US 2022/0383985 A1
(43) Pub. Date: Dec. 1, 2022

(54) MODELLING METHOD USING A CONDITIONAL VARIATIONAL AUTOENCODER

(71) Applicant: Helmholtz Zentrum Muenchen - Deutsches Forschungszentrum Fuer Gesundhelt Und Umwelt (GmbH), Neuherberg (DE)

(72) Inventors: Fabian Theis, Garching (DE); Mohammad Lotfollahi, Munich (DE); Fabian Alexander Wolf, Munich (DE)

(21) Appl. No.: 17/763,501

(22) PCT Filed: Sep. 25, 2020
(86) PCT No.: PCT/EP2020/076832

§ 371 Date: Mar. 24, 2022

(30) Foreign Application Priority Data
Sep. 25, 2019 (LU) LU101414

(51) Int. Cl. G16B 40/30; G06N 3/08; G16B 25/10

(52) U.S. Cl. CPC G16B 40/30; G06N 3/088; G16B 25/10

(57) ABSTRACT

The present invention relates to a computer-implemented method for modelling genomic data represented in an unsupervised neural network, trVAE, comprising a conditional variational autoencoder, CVAE, with an encoder (f) and a decoder (g).

### ABSTRACT OF THE PRESENT INVENTION

While generative models have shown great success in generating high-dimensional samples conditional on low-dimensional descriptors (learning e.g. stroke thickness in MNIST, hair color in CelebA, or speaker identity in Wavenet), their generation out-of-sample (or out-of-distribution) poses fundamental problems. The conditional variational autoencoder (CVAE) as a simple conditional generative model does not explicitly relate conditions during training and, hence, has no incentive of learning a compact joint distribution across conditions. We overcome this limitation by matching their distributions using maximum mean discrepancy (MMD) in the decoder layer that follows the bottleneck. This introduces a strong regularization both for reconstructing samples within the same condition and for transforming samples across conditions, resulting in much improved generalization. We refer to the architecture as transformer VAE or transfer VAE (trVAE). Benchmarking trVAE on high-dimensional image and tabular data, we demonstrate higher robustness and higher accuracy than existing approaches. In particular, we show qualitatively improved predictions for cellular perturbation response to treatment and disease based on high-dimensional single-cell gene expression data, by tackling previously problematic minority classes and multiple conditions. For generic tasks, we improve Pearson correlations of high-dimensional estimated means and variances with their ground truths from 0.89 to 0.97 and 0.75 to 0.87, respectively.

### FIELD OF THE INVENTION

The disclosed embodiments relate generally to computational modelling techniques and, more particularly, to modelling cellular perturbation response to drug treatment and disease based on high-dimensional single-cell gene expression data using a deep neural network.

While generative models have shown great success in generating high-dimensional samples conditional on low-dimensional descriptors (stroke thickness in MNIST, hair color in CelebA, speaker identity in Wavenet), their generation out-of-sample (or out-of-distribution) poses fundamental problems. The conditional variational autoencoder (CVAE) as a simple conditional generative model does not explicitly relate conditions during training and, hence, has no incentive of learning a compact joint distribution across conditions. The present invention overcomes this limitation by matching their distributions using maximum mean discrepancy (MMD) in the decoder layer that follows the bottleneck.

This introduces a strong regularization both for recon-structing samples within the same condition and for transforming samples across conditions, resulting in much improved generalization. The present invention refers to the architecture as transformer VAE or transfer VAE (trVAE). Benchmarking trVAE on high-dimensional image and tabular data, the present invention demonstrates higher robustness and higher accuracy than existing approaches. In particular, the present invention shows qualitatively improved predictions for cellular perturbation response to treatment and disease based on high-dimensional single-cell gene expression data, by tackling previously problematic minority classes and multiple conditions. For generic tasks, the present invention improves Pearson correlations of high-dimensional estimated means and variances with their ground truths from 0.89 to 0.97 and 0.75 to 0.87, respectively.

### BACKGROUND

The task of generating high-dimensional samples x conditional on a latent random vector z and a categorical variable s has established solutions (Mirza & Osindero, 2014; Ren et al., 2016). The situation becomes more complicated if the support of z is divided into different domains d with different semantic meanings: say d∈{men, women} and one is interested in out-of-sample generation of samples x in a domain and condition (d, s) that is not part of the training data. If one predicts how a given black-haired man would look with blonde hair, which we refer to as transforming x_{men, blackhair} [symbol] x_{men, blonde-hair}, this becomes an out-of-sample problem if the training data does not have instances of blonde-haired men, but merely of blonde- and black-haired woman and blacked haired men. In an application with higher relevance, there is strong interest in how untreated (s=0) humans (d=0) respond to drug treatment (s=1) based on training data from in vitro (d=1) and mice (d=2) experiments. Hence, the target domain of interest (d=0) does not offer training data for s=1, but only for s=0.

In the present paper, we suggest to address the challenge of transforming out-of-sample by regularizing the joint distribution across the categorical variable s using maximum mean discrepancy (MMD) in the framework of a conditional variational autoencoder (CVAE) (Sohn et al., 2015). This produces a more compact representation of a distribution that displays high variance in the vanilla CVAE, which incentivizes learning of features across s and results in more accurate out-of-sample prediction. MMD has proven successful in a variety of tasks. In particular, matching distributions with MMD in variational autoencoders (Kingma & Welling, 2013) has been put forward for unsupervised domain adaptation (Louizos et al., 2015) or for learning statistically independent latent dimensions (Lopez et al., 2018b). In supervised domain adaptation approaches, MMD-based regularization has been shown to be a viable strategy of learning label-predictive features with domain-specific information removed (Long et al., 2015, Tzeng et al., 2014).

In further related work, the out-of-sample transformation problem was addressed via hard-coded latent space vector arithmetics (Lotfollahi et al., 2019) and histogram matching (Amodio et al., 2018). The approach of the present paper, however, introduce a data-driven end-to-end approach, which does not involve hard-coded elements and generalizes to more than one condition.

### Variational Autoencoder

In representation learning, one aims to map a vector x to a representation z for which a given downstream task can be performed more efficiently. Hierarchical Bayesian models (Gelman & Hill, 2006) yield probalistic representations in the form of sufficient statistic for the model's posterior distribution.

Let {X,S} denote the set of observed random variables and Z the set of latent variables (Zⁱ denotes component i). Then Bayesian inference aims to maximize the likelihood:

p_{θ}(X|S)=∫p_{θ}(X|Z,S)p_{θ}(Z|S)dZ (1)

Because the integral is in general intractable, variational inference finds a distribution q_{Φ}(Z|X,S) that minimizes a lower bound on the data—the evidence lower bound (ELBO):

logₚ_{_{θ}}(X|S)≥E_{q}_{_{Φ(Z|X,S)}}[logₚ_{_{θ}}(X|Z,S)]−D_{KL}(q_{Φ}(Z|X,S)∥p(Z|S)) (2)

In the case of a variational auto-encoder (VAE), the variational distribution is parametrized by a neural network, both the generative model and the variational approximation have conditional distributions parametrized with neural networks. The difference between the data likelihood and the ELBO is the variational gap:

D_{KL}(q_{Φ}(Z|X,S)∥p_{θ}(Z|S)) (3)

The original AEVB framework is described in the seminal paper (Kingma & Welling, 2013) for the case Z={z}, X={x},S=∅. The representation z is optimized to “explain” the data x. The variational distribution can be used to meet different needs: q_{Φ}(y|x) is a classifier for a class label y and q_{Φ}(z|x) summarizes the data. When using VAE, the empirical data distribution p_{data}(X,S) is transformed to the representation \hat{q}_{Φ}(Z)=Eₚ_{_{data}}_{(x,s)}q_{Φ}(Z|X,S).

The case in which S≠∅. is referred to as the conditional variational autoencoder (CVAE) (Sohn et. al.; 2015), and a straight-forward extension of the original framework.

An alternative description of a variational autoencoder may be given as:

[symbol]

The motivation of the variational autoencoder (VAE) (Kingma and Welling, 2013) is to provide a neural-network based parametrization for maximizing the likelihood

p_{θ}(X|S)=∫p_{θ}(X|Z,S)p_{θ}(Z|S)dZ, (1)

Where X denotes a high-dimensional random variable, S a random variable representing conditions, θ the model parameters, and p_{θ}(X|Z, S) the generative distribution that decodes Z into X. Here and in the following we adapt the notation of (Lopez et al., 2018b) while adapting the presentation of (Doersch, 2016).

To assign probability mass to values of Z that are likely to produce actually observed values of X, one introduces an ancoding distribution q_{Φ}, which can be related to p_{θ} via

log p_{θ}(X|S)−(q_{ϕ}(Z|X,S)∥p_{θ}(Z|X,S))= [symbol] _{q}_{_{ϕ}}_{(Z|X,S)}[log p_{θ}(X|Z,S)]−(q_{ϕ}(Z|X,S)∥p_{θ}(Z|S)).

The right hand side of this equation provides the cost function L_{VAE}. for optimizing neural-network based parametrizations p_{θ} of and q_{Φ}. The left-hand side describes the likelihood subtracted by an error term.

The case in which S≠∅ is referred to as the conditional variational autoencoder (CVAE) (Sohn et. al.; 2015), and a straight-forward extension of the original framework (Kingma and Welling, 2013), which treated S≡∅.

### Maximum-Mean Discrepancy

Let (Ω,F,P) be a probability space. Let X (resp. X′) be a separable metric space. Let χ:Ω→X (resp. χ′:Ω→X′) be a random variable. Let k:X×X→R (resp. k:X′×X′→R) be a continuous, bounded, positive semi-definite kernel. Let H be the corresponding reproducing kernel Hilbert space (RKHS) and Φ:Ω→H the corresponding feature mapping.

Consider the kernel-based estimate of distance between two distributions p and q over the random variables X and X′. Such a distance, defined via the canonical distance between their H -embeddings is called the maximum mean discrepancy (Gretton et al., 2012) and denoted MMD(p,q), with an explicit expression:

$${ℓ}_{MMD} ( X , {X}^{′} ) = \frac{1}{{n}_{0}^{2}} {\sum }_{n , m} k ( {x}_{n} , {x}_{m} ) + \frac{1}{{n}_{1}^{2}} {\sum }_{n , m} k ( {x}_{n}^{′} , {x}_{m}^{′} ) - \frac{2}{{n}_{0} {n}_{1}} {\sum }_{n , m} k ( {x}_{n} , {x}_{m}^{′} ) , \qquad ( 4 )$$

where the sums run over the numbers of samples n₀ and n₁ for x and x′ respectively. Asymptotically, for a universal kernel such as the Gaussian kernel k(x,x′)=e^{-γ∥x-x′∥}^{²}, l_{MMD}(X,X′) is 0 if and only if p≡q.

An alternative description of maximum mean discrepancy may be given as:

[symbol]

Let (Ω,F,P) be a probability space. X a separable metric space. χ:Ω→X a random variable and k:X×X→R a continous, bounded, positive semi-definite kernel with a corresponding reproducing kernel Hilbert space (RKHS) H.

Consider the kernel-based estimate of a distance between two distributions p and q over the random variables X and X′. Such a distance, defined via the canonical distance between their H-embeddings is called the maximum mean discrepancy (Gretton et al., 2012) and denoted l_{MMD}(p, q), with an explicit expression:

$${ℓ}_{MMD} ( X , {X}^{′} ) = \frac{1}{{n}_{0}^{2}} {\sum }_{n , m} k ( {x}_{n} , {x}_{m} ) + \frac{1}{{n}_{1}^{2}} {\sum }_{n , m} k ( {x}_{n}^{′} , {x}_{m}^{′} ) - \frac{2}{{n}_{0} {n}_{1}} {\sum }_{n , m} k ( {x}_{n} , {x}_{m}^{′} ) , \qquad ( 4 ' )$$

where the sums run over the numbers of samples n₀ and n₁ for x and x′ respectively. Asymptotically, for a universal kernel such as the Gaussian kernel k(x,x′)=e^{-γ∥x-x′∥}^{²}, l_{MMD}(X,X″) is 0 if and only if p≡q.

For the implementation, we use multi-scale RBF kernels defined as:

$$k ( x , {x}^{′} ) = {{\sum }_{i = 1}}^{ℓ} k ( x , {x}^{′} , {γ}_{i} ) \qquad ( 3 )$$

### BRIEF DESCRIPTION OF THE DRAWINGS

FIG. 1 shows: The transformer VAE or transfer VAE (trVAE) is an MMD-regularized CVAE. It receives randomized batches of data (x) and condition (s) as input during training, stratified for approximately equal proportions of s. In contrast to a standard CVAE, we regularize the effect of s on the representation obtained after the first-layer g₁(\hat{z}, s) of the decoder g. During prediction time, we transform batches of the source condition xₛ₌₀ to the target condition xₛ₌₁ by encoding \hat{z}₀=f(x₀, s=0) and decoding g(\hat{z}₀, s=1).

FIG. 2 shows: Comparison of representations for MMD-layer in trVAE and the corresponding layer in the vanilla CVAE using UMAP (McInnes et al., 2018). The MMD regularization incentivizes the model to learn condition-invariant features resulting in a more compact representation. The figure shows the qualitative effect for the “PBMC data” introduced in experiments section Stimulation response. Both representations show the same number of samples.

FIG. 3 shows: Out-of-sample (or out-of-distribution) style transfer for Morpho-MNIST dataset containing normal, thin and thick digits. trVAE successfully transforms normal digits to thin (a) and thick ((b) for digits not seen during training (out-of-sample) (or out-of-distribution).

FIG. 4 shows: CelebA dataset with images in two conditions: celebrities without a smile and with a smile on their face. trVAE successfully adds a smile on faces of women without a smile despite these samples completely lacking from the training data (out-of-sample) (or out-of-distribution). The training data only comprises non-smiling women and smiling and non-smiling men

FIG. 5 shows:

(a) UMAP visualization of conditions and cell type for gut cells.

(b-c) Mean and variance expression of 1,000 genes comparing trVAE-predicted and real infected Tuft cells together with the top 10 differentially-expressed genes highlighted in red (R² denotes Pearson correlation between ground truth and predicted values).

(d) Distribution of Defa24: the top response gene to H.poly.Day10 infection between control, predicted and real stimulated cells for different models. Vertical axis: expression distribution for Defa24. Horizontal axis: control, real and predicted distribution by different models.

(e) Comparison of Pearson's R² values for mean and variance gene expression between real and predicted cells for different models. Center values show the mean of R² values estimated using n=100 random subsamples for the prediction of each model and error bars depict standard deviation.

(f) Comparison of R² values for mean gene expression between real and predicted cells by trVAE for the eight different cell types and three conditions. Center values show the mean of R² values estimated using n=100 random subsamples for each cell type and error bars depict standard deviation.

FIG. 6 shows:

(a) UMAP visualization of peripheral blood mononuclear cells (PBMCs).

(b-c) Mean and variance per 2,000 dimensions between trVAE-predicted and real natural killer cells (NK) together with the top 10 differentially-expressed genes highlighted in red.

(d) Distribution of ISG15: the most strongly changing gene after IFN-β perturbation between control, real and predicted stimulated cells for different models. Vertical axis: expression distribution for ISG15. Horizontal axis: control, real and predicted distribution by different models.

(e) Comparison of R2 values for mean and variance gene expression between real and predicted cells for different models. Center values show the mean of R2 values estimated using n=100 random subsamples for the prediction of each model and error bars depict standard deviation.

The present invention is defined by the subject matter of the appended claims.

### SUMMARY

Single-cell transcriptomics has become an established tool for unbiased profiling of complex and heterogeneous systems. The generated datasets are typically used for explaining phenotypes through cellular composition and dynamics. Of particular interest are the dynamics of single cells in response to perturbations, be it to dose, treatment or knockout of genes. Moreover, combinatorial drug treatments to cure a disease, e.g. cancers have recently been studied in single cell settings. The present invention thus provides a deep learning model as described herein which can provide in-silico predictions how combination drugs will treat the diseased samples. Previously published methods provide in-silico predictions for the scenario that only one drug exist and are not able to predict combination of drug. Moreover, previous approaches use two step modeling, first, projecting the data to a latent space and then the second algorithm performs the predictions. However, the present invention provides an end-to-end solution which does previous steps in one model. The present invention shows the performance of the model on a variety of examples as is illustrated in the examples section herein below.

Accordingly, the present invention relates to

A computer-implemented method for modelling single-cell gene expression, scGE, data represented in an unsupervised neural network, trVAE, comprising a conditional variational autoencoder, CVAE, with an encoder (f) and a decoder (g), the method comprising:

obtaining, first input data comprising one or more sets of multivariate scGE data, referred to as batches X, and one or more first conditions s associated with respective elements of said one or more sets of multivariate scGE data;

processing the first input in the encoder (f of the trVAE, thereby obtaining first latent data Z represented in a low-dimensional space of a hidden layer of the trVAE;

processing the obtained latent data Z associated with the first conditions s in a first part (g₁) of the decoder (g) thereby obtaining first reconstructed data Y;

processing the obtained first reconstructed data Y of the first layer in one or more subsequent layers in a second part (g₂) of the decoder (g), to obtain second reconstructed data \hat{ }X, thereby learning a model for reconstructing the first multivariate scGE data to facilitate curative or diagnostic interpretation,

wherein the first reconstructed data Y of the first layer are subject to a cost function derived from a known cost function L_{VAE} of the CVAE imposed with penalty based on a distance metric known as maximum mean discrepancy, MMD, or a Wasserstein distance metric.

Preferably, the computer-implemented method of the present invention further comprises the following steps carried out in the trVAE with the learned model incorporated:

obtaining second input data comprising one or more batches X of multivariate scGE data with second conditions s, wherein s=0, denoted as (Xₛ₌₀, s=0);

processing the second input in the encoder (f) of the trVAE with the learned model incorporated, thereby obtaining a second latent representation \hat{ }Z with third conditions s, wherein s=0, denoted as (\hat{ }Zₛ₌₀, s=0);

processing \hat{ }Zₛ₌₀ with fourth conditions s, wherein s=1, denoted as (\hat{ }Zₛ₌₀, s=1) in the decoder (g) of the trVAE with the learned model incorporated, to obtain transformed data \hat{ }Z associated with fourth conditions s, wherein s=1, denoted as (\hat{ }Zₛ₌₀, s=1), said transformed data representing the second multivariate scGE data associated with the fourth conditions s predicted according to the learned model.

Preferably, in the computer-implemented method of the present invention each of the first, second, third, and fourth conditions s comprises n-tupels of scalar conditions, wherein n is a natural number.

Preferably, the batches of multivariate data are randomized batches in the context of the computer-implemented method of the present invention.

Preferably, curative interpretation and diagnostic interpretation comprise predictions for cellular perturbation response to treatment and disease, respectively, based on the first multivariate scGE data in the context of the computer-implemented method of the present invention.

The methods of the present invention provide for in-silico predictions, e.g. how combination drugs will treat a diseased subject, e.g. a mammalian subject, such as a human subject.

The methods of the present invention provide for qualitatively, preferably, improved predictions for cellular perturbation response to treatment and disease based on high-dimensional single-cell gene expression data.

In the context of the present invention, genomic data may encompass data obtained from one or more transcriptomes, data obtained from one or more genomes, or data obtained from sequencing, e.g. NGS. Genomic data may be from one or more single cells, such as mammalian cells, e.g. human cells.

### DETAILED DESCRIPTION

For the implementation, we use multi-scale RBF kernels defined as:

$$k ( x , {x}^{′} ) = {{\sum }_{i = 1}}^{ℓ} k ( x , {x}^{′} , {γ}_{i} ) \qquad ( 5 )$$

where k(x,x′,γᵢ)=e^{-γi∥x-x′∥}^{²} and γᵢ is a hyper-parameter.

Addressing the domain adaptation problem, the “Variational Fair Autoencoder” (VFAE) (Louizos et al.,2015) uses MMD to match latent distributions q_{Φ}(z|s=0) and q_{Φ}(z|s=1)—where s denotes a domain—by adapting the standard VAE const function L_{VAE} according to

L_{VFAE}(Φ,θ,X,X′,S,S′)=L_{VAE}(Φ,θ,X′,S′)−βl_{MMD}(Zₛ₌₀,Z′_{s′=1}) (6)

Where X and X′ are two high-dimensional observations with their respective conditions S and S′. In contrast to GANs (Goodfellow et al., 2014), whose training procedure is notoriously hard due to the minmax optimization problem, training models using MMD or Wasserstein distance metrics is comparatively simple (Li et al., 2015; Arjovsky et al., 2017; Dziugaite et al., 2015a) as only a direct minimization of a straight forward loss is involved during the training. It has been shown that MMD based GANs have some advantages over Wasserstein GANs resulting in a simpler and faster-training algorithm with matching performance (Birikowski et al., 2018). This motivated us to choose MMD as a metric for regularizing distribution matching.

### Defining the Transformer VAE or Transfer VAE

Let us adapt the following notation for the transformation within a standard CVAE. High-dimensional observations x and a scalar or low-dimensional condition s are transformed using f (encoder) and g (decoder), which are parametrized by weight-sharing neural networks, and give rise to predictors \hat{z}, ŷ and \hat{x}.

\hat{z}=f(x,s) (7a)

ŷ=g₁(\hat{z},s) (7b)

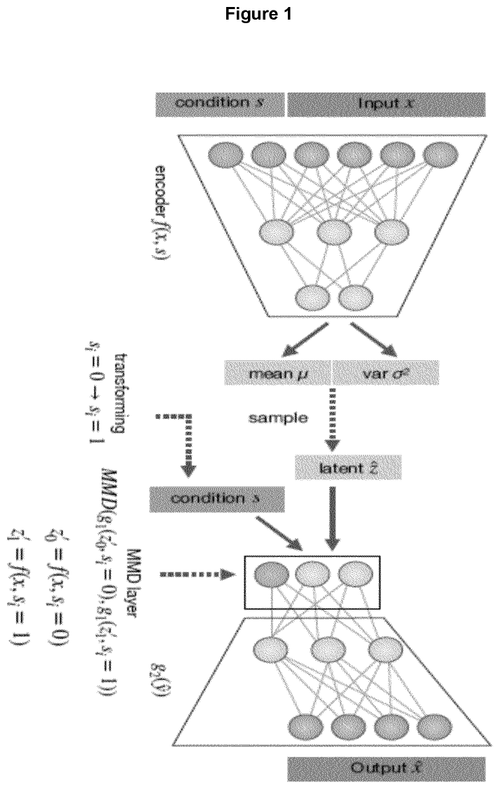

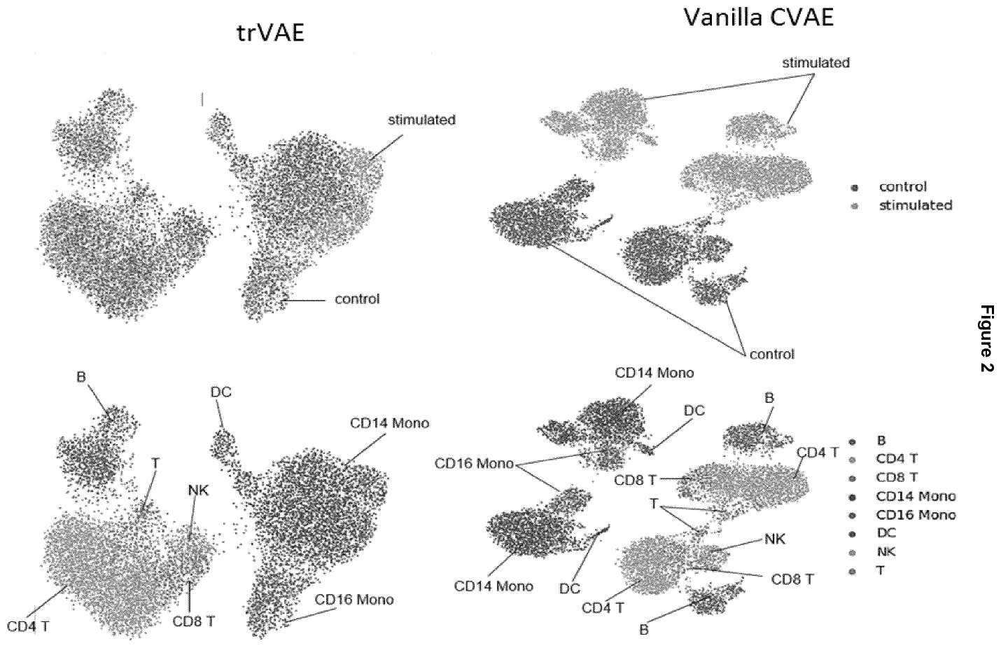

\hat{x}=g₂(\hat{y}) (7c)

where we distinguished the first (g₁) and the remaining layers (g₂) of the decoder g=g₂∘g₁ (FIG. 1).

While z formally depends on s, it is commonly empirically observed Z∥S, that is, the representation z is disentangled from the condition information s. By contrast, the original representation typically strongly covaries with S:X [symbol] S. The observation can be explained by admitting that an efficient z-representation, suitable for minimizing reconstruction and regularization losses, should be as free as possible from information about s. Information about s is directly and explicitly available to the decoder (equation 7b), and hence, there is an incentive to optimize the parameters of f to only explain the variation in x that is not explained by s. Experiments below demonstrate that indeed, MMD regularization on the bottleneck layer z does not improve performance.

However, even if z is completely free of variation from s, the y representation has a strong s component, Y [symbol] S, which leads to a separation of yₛ₌₁ and yₛ₌₀ into different regions of their support [symbol] . In the standard CVAE, without any regularization of this y representation, a highly varying, non-compact distribution emerges across different values of s (FIG. 2). To compactify the distribution so that it displays only subtle, controlled differences, we impose MMD (equation 4) in the first layer of the decoder (FIG. 1). We assume that modeling y in the same region of the support of [symbol] across s forces learning common features across s where possible. The more of these common features are learned, the more accurately the transformation task will performed, and the higher are chances of successful out-of-sample generation. Using one of the benchmark datasets introduced, below, we qualitatively illustrate the effect (FIG. 2).

During training time, all samples are passed to the model with their corresponding condition labels (xₛ,S). At prediction time, we pass (xₛ₌₀,s=0) to the encoder f to obtain the latent representation \hat{z}ₛ₌₀. In the decoder g, we pass (\hat{z}ₛ₌₀,s=1) and through that, let the model transform data to \hat{x}ₛ₌₁.

The cost function of trVAE derives directly from the standard CVAE cost function, as introduced in the backgrounds section,

L_{CVAE}(Φ,θ,X,α,η)=ηE_{qθ(Z|X,S)}log(p_{Φ}(X|Z,S))−αD_{KL} (q_{θ}(Z|X,S)∥p_{Φ}(Z|X,S)) (8)

Consistent with the above, let ŷₛ₌₀=g₁(f(x,s=0) and ŷₛ₌₁=g₁(f(x′,s=1), s=1). Through duplicating the cost function for X′ and adding an MMD term, the loss of trVAE becomes:

L_{trVAE}(Φ,θ,X,X′S,S′,α,η,β)=L_{CVAE}(Φ,θ,X,X′,S,S′,α,η)−βl_{MMD}(Ýₛ₌₀,Ŷ_{s′=1}) (9)

### EXPERIMENTS

We demonstrate the advantages of an MMD-regularized first layer of the decoder by benchmarking versus a variety of existing methods and alternatives:
Vanilla CVAE (Sohn et al., 2015)
CVAE with MMD on bottleneck (MMD-CVAE), similar to VFAE (Louizos et al., 2015)
MMD-regularized autoencoder (Dziugaite et al., 2015b; Amodio et al., 2019)
CycleGAN (Zhu et al., 2017)
scGen, a VAE combined with vector arithmetics (Lotfollahi et al., 2019)
scVl, a CVAE with a negative binomial output distribution (Lopez et al., 2018a)

First, we demonstrate trVAE's basic out-of-sample style transfer capacity on two established image datasets, on a qualitative level. We then address quantitative comparisons of challenging benchmarks with clear ground truth, predicting the effects of biological perturbation based on high-dimensional structured data. We used convolutional layers for imaging examples in section 4.1 and fully connected layers for single-cell gene expression datasets in sections 4.2 and 4.3. The optimal hyper-parameters for each application were chosen by using a parameter gird-search for each model. The detailed hyper-parameters for different models are reported in tables 1-9 in appendix A.

### MNIST and Celebra Style Transformation

Here, we use Morpho-MNIST (Castro et al., 2018) which contains 60,000 images each of _{“}normal” and _{“}transformed” digits, which are drawn with a thinner and thicker stroke. For training, we used all normal-stroke data. Hence, the training data covers all domains (d∈{0, 1, 2, . . . ,9 }) in the normal stroke condition (s=0). In the transformed conditions (thin and thick strokes, s∈{1,2}), we only kept domains d∈{1, 3, 6, 7}.

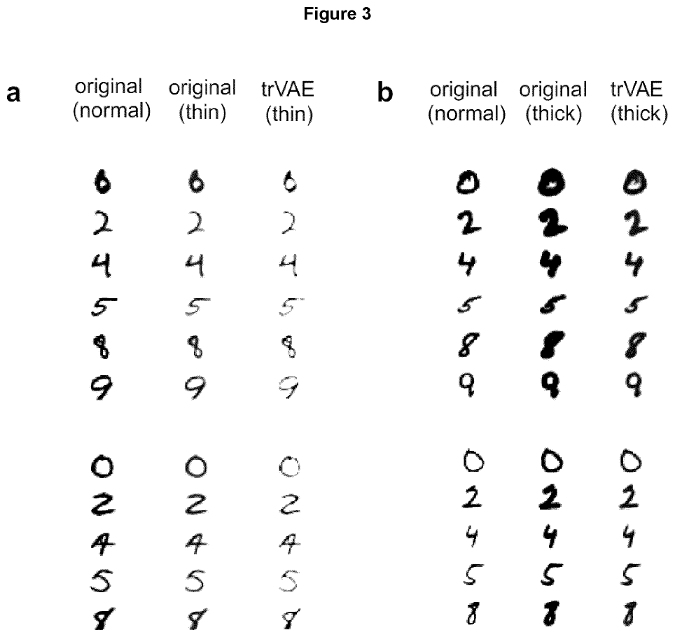

We train a convollutional trVAE in which we first encode the stroke width via two fully-connected layers with 128 and 784 features, respectively. Next, we reshape the 784-dimensional into 28*28*1 images and add them as another channel in the image. Such trained trVAE faithfully transfroms digits of normal stroke to digits of thin and thicker stroke to the out-of-sample domains (FIG. 3)

Next we apply trVAE to CelebA (Liu et al., 2015), which contains 202,599 images of celebrity faces with 40 binary attributes for each image. We focus on the task of learning a transformation that turns a non-smiling face into a smiling face. We kept the smiling (s) and gender (d) attributes and trained the model with images from both smiling and non-smiling men but only with non-smiling women.

In this case, we trained a deep convolutional trVAE with a U-Net-like architecture (Ronneberger et al., 2015). We encoded the binary condition labels as in the Morpho-MNIST example and fed them as an additional channel in the input.

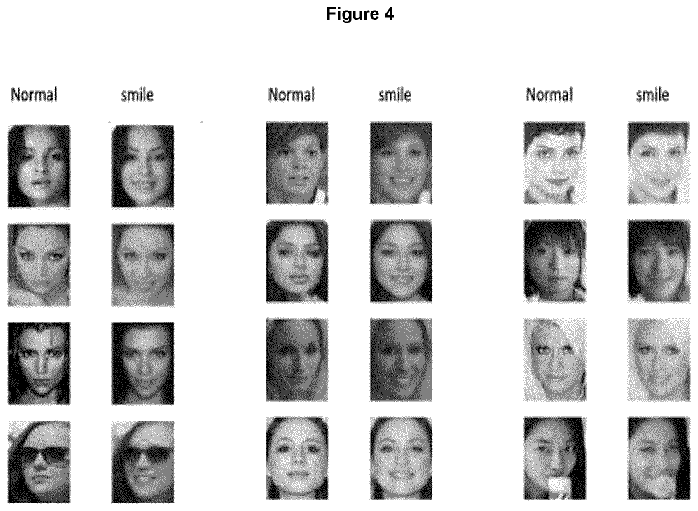

Predicting out-of-sample, trVAE successfully transforms non-smiling faces of women to smiling faces while preserving most aspects of the original image (FIG. 4). In addition to showing the model' s capacity to handle more complex data, this example demonstrates the flexibility of the model adapting to well-known architectures like U-Net in the field.

### Infection Response

Accurately modeling cell response to pertubations is a key question in computational biology. Recently, neural network models have been proposed for out-of-sample predictions of high-dimensional tabular data that quantifies gene expression of single-cells (Loffollahi et al., 2019; Amodio et al., 2018). However, these models are not trained on the task relying instead on hard-coded transformations and cannot handle more than two conditions.

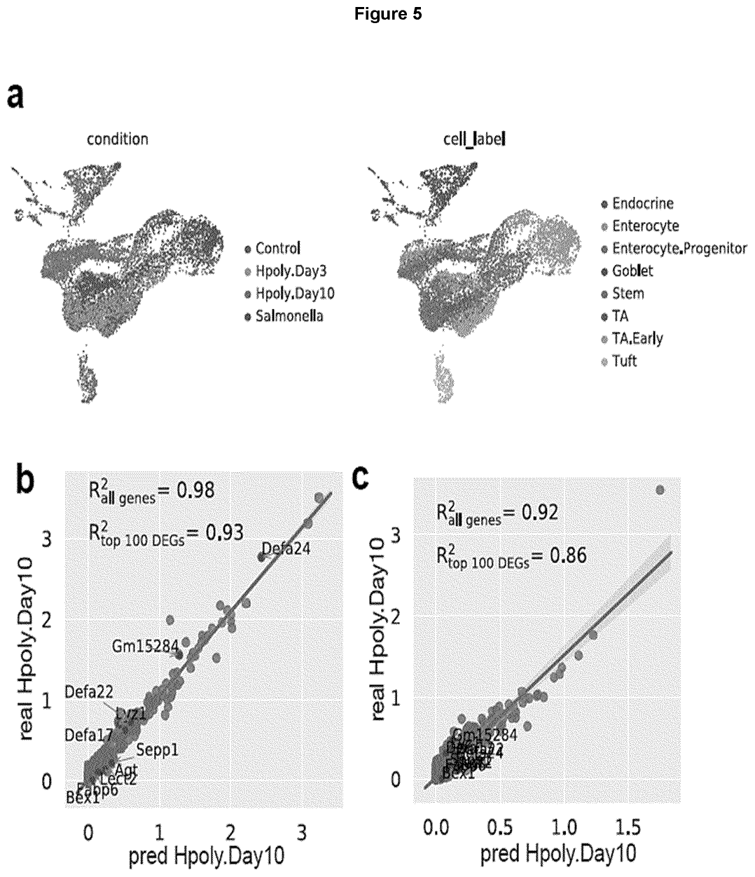

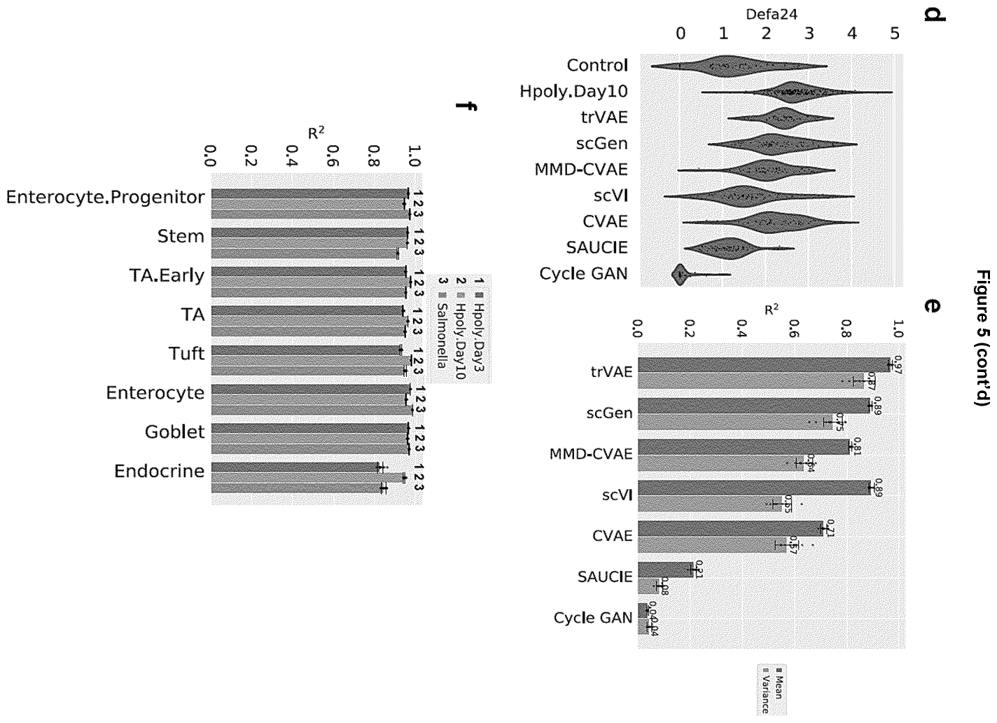

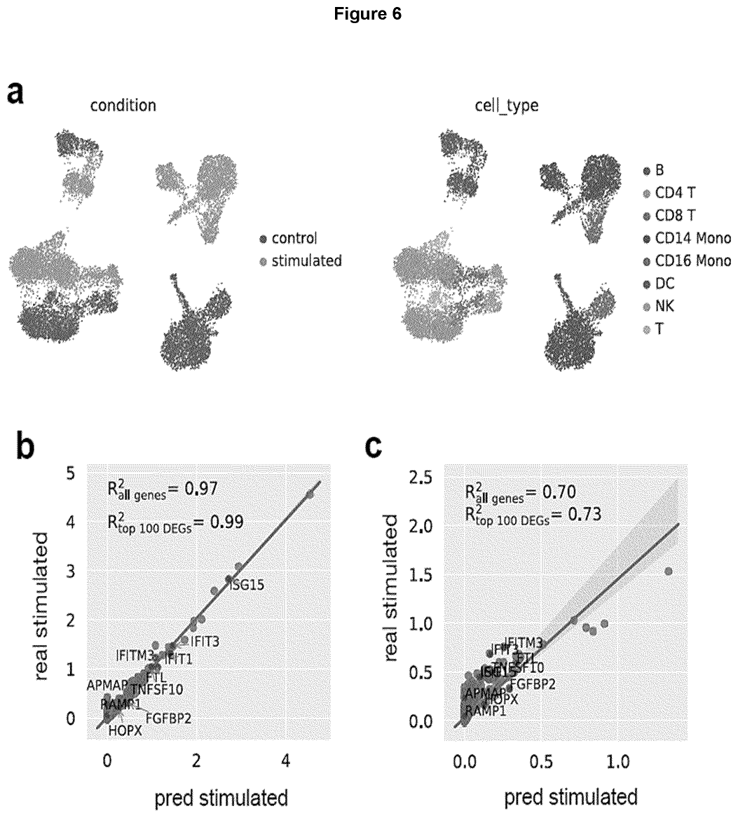

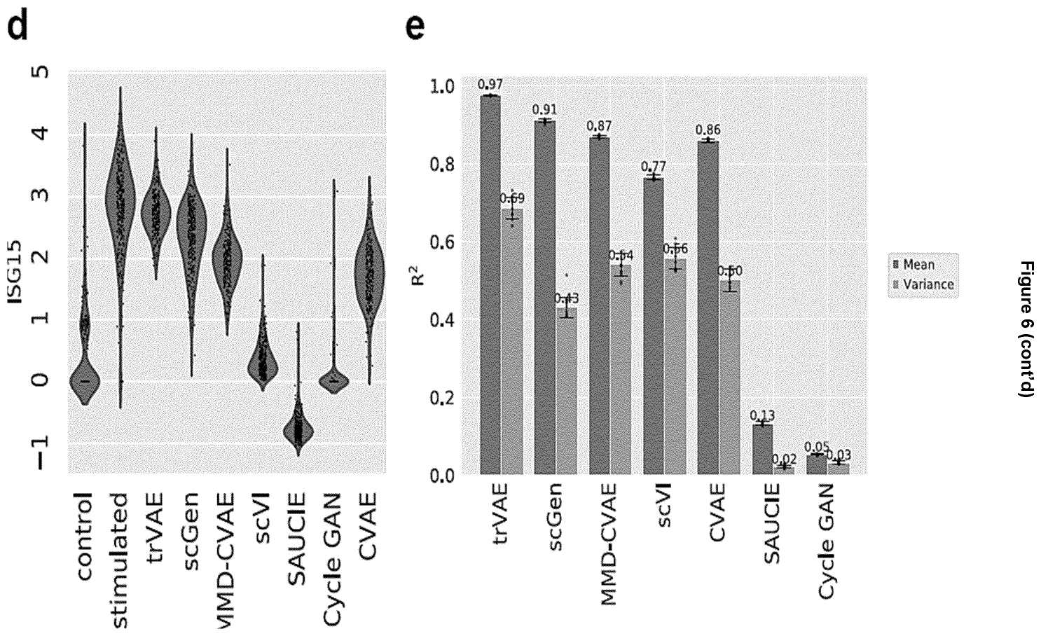

We evaluate trVAE on a single-cell gene expression dataset that characterizes the gut (Haber et al., 2017) after Salmonella or Heligmosomoides polygyrus (H. poly) infections, respectively. For this, we closely follow the benchmark as introduced in (Lotfollahi et al., 2019). The dataset contains eight different cell types in four conditions: control or healthy cells (n=3,240), H.Poly infection a after three days (H.Poly.Day3, n=2,121), H.poly infection after 10 days (H.Poly.Day10, n=2,711) and salmonella infection (n=1,770) (FIG. 5a). The normalized gene expression data has 1,000 dimensions corresponding to 1,000 genes. Since three of the benchmark models are only able to handle two conditions, we only included the control and H.Poly.Day10 conditions for model comparisons. In this settings, we hold out Tuft infected cells for training and validation, as these constitute the hardest case for out-of-sample generalization (least shared features, few training data).

FIG. 5b-c shows trVAE accurately predicts the mean and variance for high-dimensional gene expression in Tuft cells. We compared the distribution of Defa24, the gene with the highest change after H.poly infection in Tuft cells, which shows trVAE provides better estimates for mean and variance compared to other models. Moreover, trVAE outperforms other models also when quantifying the correlation of the predicted 1,000 dimensional x with its ground truth (FIG. 5e). In particular, we note that the MMD regularization on the bottleneck layer of the CVAE does not improve performance, as argued above.

In order to show our model is able to handle multiple conditions, we performed another experiment with all three conditions included. We trained trVAE holding out each of the eight cells types in all perturbed conditions. FIG. 5f shows trVAE can accurately predict all cell types in each perturbed condition, in contrast to existing models.

Applying trVAE on gut after infection show the effectiveness of MMD regularization on the first layer of the decoder and proposed architectures, allowing to model multiple drugs at the same time. The ability to analyze and predict multiple perturbations allow trVAE to be applied to experiments with many biological conditions. Specifically, recent advances in massive single-cell compounds screening (Srivatsan et al., 2020) provide great potential to exploit our model for further experimental design and the study of interaction effects among different drugs leading to advance in computational drug discovery.

Modelling multiple drugs may be carried out by applying trVAE, for example in a scenario involving two drugs:

Drug A which has condition s=[1, 0, 0], and Drug B with condition, s=[0, 1, 0] and finally control condition without any drugs with s=[0, 0, 1], wherein conditions s refer to condition labels in FIG. 1.

After training the model, the combination of A+B can be tested by feeding a control cell to the encoder with \hat{z}=f(X,S=[0, 0, 1]).

Next, \hat{z} can be fed to MMD layer (Y) with s=[1, 1, 0] which means transferring this cell into a condition where both drug A and B have been applied: ŷ=g₁(\hat{z},s=[1, 1, 0]).

Finally, \hat{x} which is the prediction of drug combination of A+B can be achieved by: \hat{x}=g₂(ŷ).

### Stimulation Response

Similar to modeling infection response as above, we benchmark on another single-cell gene expression dataset consisting of 7,217 IFN-β stimulated and 6,359 control peripheral blood mononuclear cells (PBMCs) from eight different human Lupus patients (Kang et al., 2018). The stimulation with IFN-β induced dramatic changes in the transcriptional profiles of immune cells, which causes big shifts between control and stimulated cells (FIG. 6a). We studied the out-of-sample prediction of natural killer (NK) cells held out during the training of the model.

trVAE accurately predicts mean (FIG. 6b) and variance (FIG. 6c) for all genes in the held out NK cells. In particular, genes strongly responding to IFN-β (highlighted in red in FIG. 6b-c) are well captured. An effect of applying IFN-β is an increase in ISG15 for NK cells, which the model never sees during training. trVAE predicts this change by increasing the expression of ISG15 as observed in real NK cells (FIG. 6d) A cycle GAN and an MMD-regularized auto-encoder (SAUCIE) and other models yield less accurate results than our model. Comparing the correlation of predicted mean and variance of gene expression for all dimensions of the data, we find trVAE performs best (FIG. 6e).

As shown in FIG. 2 the presence of MMD layer in the decoder layer Y that follows the bottleneck enabled a compact distribution in Y layer resulting to match distribution between control and perturbed cells. This leads to superior performance of trVAE compared to other models without MMD layer in the first layer of decoder in both mean and variance predication (FIG. 6e).

### Summary

By arguing that the vanilla CVAE yields representations in the first layer following the bottleneck that vary strongly across categorical conditions, we introduced an MMD regularization that forces these representations to be similar across conditions. The resulting model (trVAE) outperforms existing modeling approaches on benchmark and real-world data sets.

Within the bottleneck layer, CVAEs already display a well-controlled behavior, and regularization does not improve performance. Further regularization at later layers might be beneficial but is numerically costly and unstable as representations become high-dimensional. However, we have not yet systematically investigated this and leave it for future studies.

Further future work will concern the application of trVAE on larger and more data, focusing on interaction effects among conditions. For this, an important application is the study of drug interaction effects, as previously noted by Amodio et al. (2018). Future conceptual investigations concern establishing connections to causal-inference-inspired models such as CEVAE (Louizos et al., 2017): faithful modeling of an interventional distribution might possibly be re-framed as successful pertubation effect prediction across domains.

### REFERENCES

Matthew Amodio, David van Dijk, Ruth Montgomery, Guy Wolf, and Smita Krishnaswamy. Out-of-sample extrapolation with neuron editing. arXiv:1805.12198, 2018.

Matthew Amodio, David van Dijk, Krishnan Srinivasan, William S Chen, Hussein Mohsen, Kevin R. Moon, Allison Campbell, Yujiao Zhao, Xiaomei Wang, Manjunatha Venkataswamy, Anita Desai, V. Ravi, Priti Kumar, Ruth Montgomery, Guy Wolf, and Smita Krishnaswamy. Exploring single-cell data with deep multitasking neural networks. bioRxiv, 2019. doi: 10.1101/237065.

Martin Arjovsky, Soumith Chintala, and Leon Bottou. Wasserstein generative adversarial networks. In Doina Precup and Yee Whye Teh (eds.), Proceedings of the 34th International Conference on Machine Learning, volume 70 of Proceedings of Machine Learning Research, pp. 214-223, International Convention Centre, Sydney, Australia, 6-11 Aug. 2017. PMLR.

Mikalaj Birikowski, Dougal J Sutherland, Michael Arbel, and Arthur Gretton. Demystifying mmd gans. arXiv:1801.01401, 2018.

Daniel C. Castro, Jeremy Tan, Bernhard Kainz, Ender Konukoglu, and Ben Glocker. Morpho-MNIST: Quantitative assessment and diagnostics for representation learning. 2018.

Gintare Karolina Dziugaite, Daniel M. Roy, and Zoubin Ghahramani. Training generative neural networks via maximum mean discrepancy optimization. In Proceedings of the Thirty-First Conference on Uncertainty in Artificial Intelligence, UAI′15, pp. 258-267, Arlington, Va., United States, 2015a. AUAI Press.

Gintare Karolina Dziugaite, Daniel M Roy, and Zoubin Ghahramani. Training generative neural networks via maximum mean discrepancy optimization. arXiv:1505.03906, 2015b.

Andrew Gelman and Jennifer Hill. Data analysis using regression and multilevel/hierarchical models. Cambridge university press, 2006.

Ian Goodfellow, Jean Pouget-Abadie, Mehdi Mirza, Bing Xu, David Warde-Farley, Sherjil Ozair, Aaron Courville, and Yoshua Bengio. Generative adversarial nets. In Advances in neural information processing systems, pp. 2672-2680, 2014.

Arthur Gretton, Karsten M Borgwardt, Malte J Rasch, Bernhard Schölkopf, and Alexander Smola. A kernel two-sample test. Journal of Machine Learning Research, 13:723-773, 2012.

Adam L. Haber, Moshe Biton, Noga Rogel, Rebecca H. Herbst, Karthik Shekhar, Christopher Smillie, Grace Burgin, Toni M. Delorey, Michael R. Howitt, Yarden Katz, Itay Tirosh, Semir Beyaz, Danielle Dionne, Mei Zhang, Raktima Raychowdhury, Wendy S. Garrett, Orit Rozenblatt-Rosen, Hai Ning Shi, Omer Yilmaz, Ramnik J. Xavier, and Aviv Regev. A single-cell survey of the small intestinal epithelium. Nature, 551:333,2017.

Hyun Min Kang, Meena Subramaniam, Sasha Targ, Michelle Nguyen, Lenka Maliskova, Elizabeth McCarthy, Eunice Wan, Simon Wong, Lauren Byrnes, Cristina M Lanata, et al. Multiplexed droplet single-cell rna-sequencing using natural genetic variation. Nature biotechnology, 36(1):89, 2018.

Diederik P Kingma and Max Welling. Auto-encoding variational bayes. arXiv:1312.6114, 2013. Yujia Li, Kevin Swersky, and Rich Zemel. Generative moment matching networks. In International Conference on Machine Learning, pp. 1718-1727, 2015.

Ziwei Liu, Ping Luo, Xiaogang Wang, and Xiaoou Tang. Deep learning face attributes in the wild.

In Proceedings of International Conference on Computer Vision (ICCV), December 2015.

Mingsheng Long, Yue Cao, Jianmin Wang, and Michael I Jordan. Learning transferable features with deep adaptation networks. arXiv:1502.02791,2015.

Romain Lopez, Jeffrey Regier, Michael B. Cole, Michael I. Jordan, and Nir Yosef. Deep generative modeling for single-cell transcriptomics. Nature Methods, 15(12):1053-1058, 2018a.

Romain Lopez, Jeffrey Regier, Michael I Jordan, and Nir Yosef. Information constraints on auto-encoding variational bayes. In Advances in Neural Information Processing Systems, pp. 6114-6125, 2018b.

Mohammad Lotfollahi, F Alexander Wolf, and Fabian J Theis. scGen predicts single-cell perturbation responses. Nature methods, 16(8):715, 2019.

Christos Louizos, Kevin Swersky, Yujia Li, Max Welling, and Richard Zemel. The variational fair autoencoder. arXiv:1511.00830, 2015.

Christos Louizos, Uri Shalit, Joris M Mooij, David Sontag, Richard Zemel, and Max Welling. Causal effect inference with deep latent-variable models. In Advances in Neural Information Processing Systems, pp. 6446-6456, 2017.

L. McInnes, J. Healy, and J. Melville. UMAP: Uniform Manifold Approximation and Projection for Dimension Reduction. arXiv:1802.03426, 2018.

Mehdi Mirza and Simon Osindero. Conditional generative adversarial nets. arXiv:1411.1784, 2014. Yong Ren, Jun Zhu, Jialian Li, and Yucen Luo. Conditional generative moment-matching networks.

In Advances in Neural Information Processing Systems, pp. 2928-2936, 2016.

Olaf Ronneberger, Philipp Fischer, and Thomas Brox. U-net: Convolutional networks for biomedical image segmentation. Medical Image Computing and Computer-Assisted Intervention—MICCAI 2015, pp. 234-241, 2015.

Kihyuk Sohn, Honglak Lee, and Xinchen Yan. Learning structured output representation using deep conditional generative models. In Advances in Neural Information Processing Systems 28, pp. 3483-3491. 2015.

Eric Tzeng, Judy Hoffman, Ning Zhang, Kate Saenko, and Trevor Darrell. Deep domain confusion: Maximizing for domain invariance. arXiv:1412.3474, 2014.

Jun-Yan Zhu, Taesung Park, Phillip Isola, and Alexei A Efros. Unpaired image-to-image translation using cycle-consistent adversarial networks. In IEEE International Conference on Computer Vision (ICCV), 2017.

Erasian et al., “Deep learning: new computational modelling techniques for genomics”; https://doi.orq/10.1038/s41576-019-0122-6

Goodfellow, I., Bengio, Y. & Courville, A. Deep Learning (MIT Press, 2016).

Doersch (2016). Tutorial on variational autoencoders. arXiv: 1606.05908.

TABLE 1
Convolutional trVAE detailed architecture used for Morpho-MNIST dataset.

| Name | Operation | NoF/Kernel Dim. | Dropout | BN | Activation | Input |
|---|---|---|---|---|---|---|
| input | — | (28, 28, 1) | X | X | — | — |
| conditions | — | 2 | X | X | — | — |
| FC-1 | FC | 128 | | X | Leak [symbol] ReLU | conditions |
| FC-2 | FC | 784 | 0.2 | | Leaky ReLU | FC-1 |
| FC-2_resh | Reshape | (28, 28, 1) | X | X | X | FC-2 |
| Conv2D_1 | Conv2D | (4, 4, 64, 2) | X | X | Leaky ReLU | [FC-2_resh, input] |
| Conv2D_2 | Conv2D | (4, 4, 64, 64) | X | X | Leak [symbol] ReLU | Conv2D_1 |
| FC-3 | FC | 128 | X | | Leak [symbol] ReLU | Flatten(Conv2D_2) |
| mean | FC | 50 | X | X | Linear | FC-3 var FC 50 X |
| | X | Linear | FC-3 | | | |
| z | FC | 50 | X | X | Linear | [mean, var] |
| FC-4 | FC | 128 | | X | Leak [symbol] ReLU | conditions |
| FC-5 | FC | 784 | 0.2 | | Leaky ReLU | FC-4 |
| FC-5_resh | Reshape | (28, 28, 1) | X | X | X | FC-5 |
| MMD | FC | 128 | X | | Leak [symbol] ReLU | [z, FC-5_resh] |
| FC-6 | FC | 256 | | X | Leak [symbol] ReLU | MMD |
| FC-7_resh | Reshape | (2, 2, 64) | | X | X X | FC-6 |
| Conv_transp_1 | Conv2D Transpose | (4, 4, 128, 64) | | X | Leak [symbol] ReLU | FC-7_resh |
| Conv_transp_2 | Conv2D Transpose | (4, 4, 64, 64) | | X | Leak [symbol] ReLU | UpSampling2D(Conv_tr |
| Conv_transp_3 | Conv2D Transpose | (4, 4, 64, 64) | | X | Leak [symbol] ReLU | Conv_transp_2 |
| Conv_transp_4 | Conv2D Transpose | (4, 4, 2, 64) | | X | Leak [symbol] ReLU | UpSampling2D(Conv_tr |
| output | Conv2D Transpose | (4, 4, 1, 2) | | X | ReL [symbol] | UpSampling2D(Conv_tr Optimizer Adam |
| Learning Rate | 0.001 | | | | | |
| Leaky ReLU slope | 0.2 | | | | | |
| Batch Size | 1024 | | | | | |
| # of Epochs | 5000 | | | | | |
| α | 0.001 | | | | | |
| β | 1000 | | | | | |

[symbol]

indicates data missing or illegible when filed

TABLE 2
U-Net trVAE detailed architecture used for CelebA dataset.

| Name | Operation | NoF/Kernel Dim. | Dropout | BN | Activation | Input |
|---|---|---|---|---|---|---|
| input | — | (64, 64, 3) | X | X | — | — |
| conditions | — | 2 | X | X | — | — |
| FC-1 | FC | 128 | X | [symbol] | ReLU | conditions |
| FC-2 | FC | 1024 | 0.2 | [symbol] | ReLU | FC-1 |
| FC-2_reshaped | Reshape | (64, 64, 1) | X | X | X | FC-2 |
| Conv_1 | Conv2D | (3, 3, 64, 4) | X | X | ReLU | [FC-2_reshaped, input] |
| Conv_2 | Conv2D | (3, 3, 64, 64) | X | X | ReLU | Conv_1 |
| Pool_1 | Pooling2D | X | X | X | X | Conv_2 |
| Conv_3 | Conv2D | (3, 3, 128, 64) | X | X | ReLU | Pool_1 |
| Conv_4 | Conv2D | (3, 3, 128, 128) | X | X | ReLU | Conv_3 |
| Pool_2 | Pooling2D | X | X | X | X | Conv_4 |
| Conv_5 | Conv2D | (3, 3, 256, 128) | X | X | ReLU | Pool_2 |
| Conv_6 | Conv2D | (3, 3, 256, 256) | X | X | ReLU | Conv_5 |
| Conv_7 | Conv2D | (3, 3, 256, 256) | X | X | ReLU | Conv_6 |
| Pool_3 | Pooling2D | X | X | X | X | Conv_7 |
| Conv_8 | Conv2D | (3, 3, 512, 256) | X | X | ReLU | Pool_3 |
| Conv_9 | Conv2D | (3, 3, 512, 512) | X | X | ReLU | Conv_8 |
| Conv_10 | Conv2D | (3, 3, 512, 512) | X | X | ReLU | Conv_9 |
| Pool_4 | Pooling2D | X | X | X | X | Conv_10 |
| Conv_11 | Conv2D | (3, 3, 512, 256) | X | X | ReLU | Pool_4 |
| Conv_12 | Conv2D | (3, 3, 512, 512) | X | X | ReLU | Conv_11 |
| Conv_13 | Conv2D | (3, 3, 512, 512) | X | X | ReLU | Conv_12 |
| Pool_4 | Pooling2D | X | X | X | X | Conv_13 |
| flat | Flatten | X | X | X | X | Pool_4 |
| FC-3 | FC | 1024 | X | X | ReLU | flat |
| FC-4 | FC | 256 | 0.2 | X | ReLU | FC-3 |
| mean | FC | 60 | X | X | Linear | FC-4 |
| var | FC | 60 | X | X | Linear | FC-4 |
| z-sample | FC | 60 | X | X | Linear | [mean, var] |
| FC-5 | FC | 128 | X | [symbol] | ReLU | conditions |
| MMD | FC | 256 | X | [symbol] | ReLU | [z-sample, FC-5] |
| FC-6 | FC | 1024 | X | X | ReLU | MMD |
| FC-7 | FC | 4096 | X | X | ReLU | FC-6 |
| FC-7_reshaped | Reshape | X | X | X | FC-7 | |
| Conv_transp_1 | Conv2D Transpose | (3, 3, 512, 512) | X | X | ReLU | FC-7_reshaped |
| Conv_transp_2 | Conv2D Transpose | (3, 3, 512, 512) | X | X | ReLU | Conv_transp_1 |
| Conv_transp_3 | Conv2D Transpose | (3, 3, 512, 512) | X | X | ReLU | Conv_transp_2 |
| up_sample_1 | UpSampling2D | X | X | X | X | Conv_transp_3 |
| Conv_transp_4 | Conv2D Transpose | (3, 3, 512, 512) | X | X | ReLU | up_sample_1 |
| Conv_transp_5 | Conv2D Transpose | (3, 3, 512, 512) | X | X | ReLU | Conv_transp_4 |
| Conv_transp_6 | Conv2D Transpose | (3, 3, 512, 512) | X | X | ReLU | Conv_transp_5 |
| up_sample_2 | UpSampling2D | X | X | X | X | Conv_transp_6 |
| Conv_transp_7 | Conv2D Transpose | (3, 3, 128, 256) | X | X | ReLU | up_sample_2 |
| Conv_transp_8 | Conv2D Transpose | (3, 3, 128, 128) | X | X | ReLU | Conv_transp_7 |
| up_sample_3 | UpSampling2D | X | X | X | X | Conv_transp_8 |
| Conv_transp_9 | Conv2D Transpose | (3, 3, 64, 128) | X | X | ReLU | up_sample_3 |
| Conv_transp_10 | Conv2D Transpose | (3, 3, 64, 64) | X | X | ReLU | Conv_transp_9 |
| output | Conv2D Transpose | (1, 1, 3, 64) | X | X | ReLU | Conv_transp_10 |
| Optimizer | Adam | | | | | |
| Learning Rate | 0.001 | | | | | |
| Leaky ReLU slope | 0.2 | | | | | |
| Batch Size | 1024 | | | | | |
| # of Epochs | 5000 | | | | | |
| α | 0.001 | | | | | |
| β | 1000 | | | | | |

[symbol]

indicates data missing or illegible when filed

TABLE 3
trVAE detailed architecture. We used the same architecture for all the examples in the
paper. The input_dim parameter for each dataset is: IFN-β (2,000), H.poly (1,000).

| Name | Operation | NoF/Kernel Dim. | Dropout | BN | Activation | Input |
|---|---|---|---|---|---|---|
| input | — | input_dim | X | X | — | — |
| conditions | — | n_conditions | X | [symbol] | — | — |
| FC-1 | FC | 800 | 0.2 | [symbol] | Leaky ReLU | [input, conditions] |
| FC-2 | FC | 800 | 0.2 | ✓ | Leaky ReLU | FC-1 |
| FC-3 | FC | 128 | 0.2 | ✓ | Leaky ReLU | FC-2 |
| mean | FC | 50 | X | X | Linear | FC-3 |
| var | FC | 50 | X | X | Linear | FC-3 |
| z-sample | FC | 50 | X | [symbol] | Linear | [mean, var] |
| MMD | FC | 128 | 0.2 | [symbol] | Leaky ReLU | [z-sample, conditions] |
| FC-4 | FC | 800 | 0.2 | ✓ | Leaky ReLU | MMD |
| FC-5 | FC | 800 | 0.2 | ✓ | Leaky ReLU | FC-3 |
| output | FC | input_dim | X | X | ReLU | FC-4 |
| Optimizer | Adam | | | | | |
| Learning Rate | 0.001 | | | | | |
| Leaky ReLU slope | 0.2 | | | | | |
| Batch Size | 512 | | | | | |
| # of Epochs | 5000 | | | | | |
| α | 0.00001 | | | | | |
| β | 100 | | | | | |
| η | 100 | | | | | |

[symbol]

indicates data missing or illegible when filed

TABLE 4
scGen detailed architecture.

| Name | Operation | NoF/Kernel Dim. | Dropout | BN | Activation | Input |
|---|---|---|---|---|---|---|
| input | — | input_dim | X | [symbol] | — | — |
| FC-1 | FC | 800 | 0.2 | [symbol] | Leaky ReLU | input |
| FC-2 | FC | 800 | 0.2 | ✓ | Leaky ReLU | FC-1 |
| FC-3 | FC | 128 | 0.2 | ✓ | Leaky ReLU | FC-2 |
| mean | FC | 100 | X | X | Linear | FC-3 |
| var | FC | 100 | X | X | Linear | FC-3 |
| z | FC | 100 | X | [symbol] | Linear | [mean, var] |
| MMD | FC | 128 | 0.2 | [symbol] | Leaky ReLU | z |
| FC-4 | FC | 800 | 0.2 | ✓ | Leaky ReLU | MMD |
| FC-5 | FC | 800 | 0.2 | ✓ | Leaky ReLU | FC-3 |
| output | FC | input_dim | X | X | ReLU | FC-4 |
| Optimizer | Adam | | | | | |
| Learning Rate | 0.001 | | | | | |
| Leaky ReLU slope | 0.2 | | | | | |
| Batch Size | 32 | | | | | |
| # of Epochs | 300 | | | | | |
| α | 0.00050 | | | | | |
| β | 100 | | | | | |
| η | 100 | | | | | |

[symbol]

indicates data missing or illegible when filed

TABLE 5
CVAE detailed architecture.

| Name | Operation | NoF/Kernel Dim. | Dropout | BN | Activation | Input |
|---|---|---|---|---|---|---|
| input | — | input_dim | X | X | — | — |
| conditions | — | 1 | X | [symbol] | — | — |
| FC-1 | FC | 800 | 0.2 | [symbol] | Leaky ReLU | [input, conditions] |
| FC-2 | FC | 800 | 0.2 | ✓ | Leaky ReLU | FC-1 |
| FC-3 | FC | 128 | 0.2 | ✓ | Leaky ReLU | FC-2 |
| mean | FC | 50 | X | X | Linear | FC-3 |
| var | FC | 50 | X | X | Linear | FC-3 |
| z-sample | FC | 50 | X | [symbol] | Linear | [mean, var] |
| MMD | FC | 128 | 0.2 | [symbol] | Leaky ReLU | [z-sample, conditions] |
| FC-4 | FC | 800 | 0.2 | ✓ | Leaky ReLU | MMD |
| FC-5 | FC | 800 | 0.2 | ✓ | Leaky ReLU | FC-3 |
| output | FC | input_dim | X | X | ReLU | FC-4 |
| Optimizer | Adam | | | | | |
| Learning Rate | 0.001 | | | | | |
| Leaky ReLU slope | 0.2 | | | | | |
| Batch Size | 512 | | | | | |
| # of Epochs | 300 | | | | | |
| α | 0.001 | | | | | |

[symbol]

indicates data missing or illegible when filed

TABLE 6
MMD-CVAE detailed architecture.

| Name | Operation | NoF/Kernel Dim. | Dropout | BN | Activation | Input |
|---|---|---|---|---|---|---|
| input | — | input_dim | X | X | — | — |
| conditions | — | 1 | X | [symbol] | — | — |
| FC-1 | FC | 800 | 0.2 | [symbol] | Leaky ReLU | [input, conditions] |
| FC-2 | FC | 800 | 0.2 | ✓ | Leaky ReLU | FC-1 |
| FC-3 | FC | 128 | 0.2 | ✓ | Leaky ReLU | FC-2 |
| mean | FC | 50 | X | X | Linear | FC-3 |
| var | FC | 50 | X | X | Linear | FC-3 |
| z-sample | FC | 50 | X | [symbol] | Linear | [mean, var] |
| MMD | FC | 128 | 0.2 | [symbol] | Leaky ReLU | [z-sample, conditions] |
| FC-4 | FC | 800 | 0.2 | ✓ | Leaky ReLU | MMD |
| FC-5 | FC | 800 | 0.2 | ✓ | Leaky ReLU | FC-3 |
| output | FC | input_dim | X | X | ReLU | FC-4 |
| Optimizer | Adam | | | | | |
| Learning Rate | 0.001 | | | | | |
| Leaky ReLU slope | 0.2 | | | | | |
| Batch Size | 512 | | | | | |
| # of Epochs | 500 | | | | | |
| α | 0.001 | | | | | |
| β | 1 | | | | | |

[symbol]

indicates data missing or illegible when filed

TABLE 7
Style transfer GAN detailed architecture.

| Name | Operation | NoF/Kernel Dim. | Dropout | BN | Activation | Input |
|---|---|---|---|---|---|---|
| input | — | input_dim | X | X | — | — |
| FC-1 | FC | 700 | 0.5 | ✓ | Leaky ReLU | input |
| FC-2 | FC | 100 | 0.5 | ✓ | Leaky ReLU | FC-1 |
| FC-3 | FC | 50 | 0.5 | ✓ | Leaky ReLU | FC-2 |
| FC-4 | FC | 100 | 0.5 | ✓ | Leaky ReLU | FC-3 |
| FC-5 | FC | 700 | 0.5 | ✓ | Leaky ReLU | FC-4 |
| generator_out | FC | 6,998 | X | ✓ | ReLU | FC-5 |
| FC-6 | FC | 700 | 0.5 | ✓ | Leaky ReLU | generator_out |
| FC-7 | FC | 100 | 0.5 | ✓ | Leaky ReLU | FC-6 |
| discriminator_out | FC | 1 | X | X | Sigmoid | FC-7 |
| Generator Optimizer | Adam | | | | | |
| Discriminator Optimizer | Adam | | | | | |
| Optimizer | Adam | | | | | |
| Learning Rate | 0.001 | | | | | |
| Leaky ReLU slope | 0.2 | | | | | |
| # of Epochs | 1000 | | | | | |

TABLE 8
scVI detailed architecture.

| Name | Operation | NoF/Kernel Dim. | Dropout | BN | Activation | Input |
|---|---|---|---|---|---|---|
| input | — | input_dim | X | X | — | — |
| conditions | — | 1 | X | X | — | — |
| FC-1 | FC | 128 | 0.2 | ✓ | ReLU | input |
| mean | FC | 10 | X | X | Linear | FC-1 |
| var | FC | 10 | X | X | Linear | FC-1 |
| z | FC | 10 | X | X | Linear | [mean, var] |
| FC-2 | FC | 128 | 0.2 | ✓ | ReLU | [z, conditions] |
| output | FC | input_dim | X | X | ReLU | FC-2 |
| Optimizer | Adam | | | | | |
| Learning Rate | 0.001 | | | | | |
| Batch Size | 128 | | | | | |
| # of Epochs | 1000 | | | | | |
| a | 0.001 | | | | | |

TABLE 9
SAUCIE detailed architecture

| Name | Operation | NoF/Kernel Dim. | Dropout | BN | Activation | Input |
|---|---|---|---|---|---|---|
| input | — | input_dim | X | X | — | — |
| conditions | — | 1 | X | X | — | — |
| FC-1 | FC | 512 | X | ✓ | Leaky ReLU | [input, conditions] |
| FC-2 | FC | 256 | X | X | Leaky ReLU | FC-1 |
| FC-3 | FC | 128 | X | X | Leaky ReLU | FC-2 |
| FC-4 | FC | 20 | X | X | Leaky ReLU | FC-3 |
| FC-5 | FC | 128 | X | X | Leaky ReLU | FC-4 |
| FC-6 | FC | 256 | X | X | Leaky ReLU | FC-5 |
| FC-7 | FC | 512 | X | X | Leaky ReLU | FC-6 |
| output | FC | input_dim | X | X | ReLU | FC-4 |
| Optimizer | Adam | | | | | |
| Learning Rate | 0.001 | | | | | |
| Leaky ReLU slope | 0.2 | | | | | |
| Batch Size | 256 | | | | | |
| # of Epochs | 1000 | | | | | |

1. A computer-implemented method for modelling single-cell gene expression, scGE, data represented in an unsupervised neural network, trVAE, comprising a conditional variational autoencoder, CVAE, with an encoder (f) and a decoder (g), the method comprising:
obtaining, first input data comprising one or more sets of multivariate scGE data, referred to as batches X, and one or more first conditions s associated with respective elements of said one or more sets of multivariate scGE data;
processing the first input in the encoder (f) of the trVAE, thereby obtaining first latent data Z represented in a low-dimensional space of a hidden layer of the trVAE;
processing the obtained latent data Z associated with the first conditions s in a first part (g₁) of the decoder (g) thereby obtaining first reconstructed data Y;
processing the obtained first reconstructed data Y of the first layer in one or more subsequent layers in a second part (g₂) of the decoder (g), to obtain second reconstructed data \hat{ }X, thereby learning a model for reconstructing the first multivariate scGE data to facilitate curative or diagnostic interpretation,
wherein the first reconstructed data Y of the first layer are subject to a cost function derived from a known cost function L_{VAE} of the CVAE imposed with penalty based on a distance metric known as maximum mean discrepancy, MMD, or a Wasserstein distance metric.

2. The method of the preceding claim, further comprising the following steps carried out in the trVAE with the learned model incorporated:
obtaining second input data comprising one or more batches X of multivariate scGE data with second conditions s, wherein s=0, denoted as (Xₛ₌₀, s=0);
processing the second input in the encoder (f) of the trVAE with the learned model incorporated, thereby obtaining a second latent representation \hat{ }Z with third conditions s, wherein s=0, denoted as (\hat{ }Zₛ₌₀, s=0); processing \hat{ }Zₛ₌₀ with fourth conditions s, wherein s=1, denoted as (\hat{ }Zₛ₌₀, s=1) in the decoder (g) of the trVAE with the learned model incorporated, to obtain transformed data \hat{ }Z associated with fourth conditions s, wherein s=1, denoted as (\hat{ }Zₛ₌₀, s=1), said transformed data representing the second multivariate scGE data associated with the fourth conditions s predicted according to the learned model.

3. The method of the two preceding claims, wherein each of the first, second, third, and fourth conditions s comprises n-tupels of scalar conditions, wherein n is a natural number.

4. The method of any of the preceding claims, wherein the batches of multivariate data are randomized batches.

5. The method of any of the preceding claims, wherein curative interpretation and diagnostic interpretation comprise predictions for cellular perturbation response to treatment and disease, respectively, based on the first multivariate scGE data.
